SEQUENCE LISTING (13 sequences)

SEQ ID NO: 1 - DNA, 45 bp, Homo sapiens
atcctccaagtcatccaactggtccgggtgttccaaatcttcaag

SEQ ID NO: 2 - DNA, 44 bp, Homo sapiens
ttgaagattggaacacccggaccagttggatgacttggaggatg

SEQ ID NO: 3 - DNA, 33 bp, Homo sapiens
attgccctgcctgtggacgtcatcgtctccaac

SEQ ID NO: 4 - DNA, 33 bp, Homo sapiens
ttggagacgatgacgtccacaggcagggcaatg

SEQ ID NO: 5 - protein, 159 aa, Homo sapiens
PYFITLGTEIAEQEGNQKGEQATSLAILRVIRLVRVFRIFKLSRHSKGLQILGQTLKASMRELGLLIFFLFIGVILFSSAVYFAEAEEAESHFSSIPDAFWWAVVSMTTVGYGDMYPVTIGGKIVGSLCAIAGVLTIALPVPVIVSNFNYFYHRETEGE

SEQ ID NO: 6 - protein, 160 aa, Mus Musculus
PYFITLGTELAEKPEDAQQGQQAMSLAILRVIRLVRVFRIFKLSRHSKGLQILGQTLKASMRELGLLIFFLFIGVILFSSAVYFAEADERDSQFPSIPDAFWWAVVSMTTVGYGDMVPTTIGGKIVGSLCAIAGVLTIALPVPVIVSNFNYFYHRETEGE

SEQ ID NO: 7 - protein, 161 aa, Homo sapiens
PYFITLGTDLAQQQGGGNGQQQQAMSFAILRIIRLVRVFRIFKLSRHSKGLQILGHTLRASMRELGLLIFFLFIGVILFSSAVYFAEADEPTTHFQSIPDAFWWAVVTMTTVGYGDMKPITVGGKIVGSLCAIAGVLTIALPVPVIVSNFNYFYHRETENE

SEQ ID NO: 8 - protein, 157 aa, Homo sapiens
PYFITLGTELAERQGNGQQAMSLAILRVIRLVRVFRIFKLSRHSKGLQILGQTLKASMRELGLLIFFLFIGVILFSSAVYFAEADDPTSGFSSIPDAFWWAVVTMTTVGYGDMHPVTIGGKIVGSLCAIAGVLTIALPVPVIVSNFNYFYHRETEGE

SEQ ID NO: 9 - protein, 164 aa, Homo sapiens
PYFITLGTELAEQQPGGGGGGQNGQQAMSLAILRVIRLVRVFRIFKLSRHSKGLQILGKTLQASMRELGLLIFFLFIGVILFSSAVYFAEADNQGTHFSSIPDAFWWAVVTMTTVGYGDMRPITVGGKIVGSLCAIAGVLTIALPVPVIVSNFNYFYHRETDHE

SEQ ID NO: 10 - protein, 171 aa, Drosophila melagaster
PYFITLATVVAEEEDTLNLPKAPVSPQDKSSNQAMSLAILRVIRLVRVFRIFKLSRHSKGLQILGRTLKASMRELGLLIFFLFIGVVLFSSAVYFAEAGSENSFFKSIPDAFWWAVVTMTTVGYGDMTPVGVWGKIVGSLCAIAGVLTIALPVPVIVSNFNYFYHRETDQE

SEQ ID NO: 11 - protein, 163 aa, Rattus norvegicus
PFYLEVGLSGLSSKAAKDVLGFLRVVRFVRILRIFKLTRHFVGLRVLGHTLRASTNEFLLLIIFLALGVLIFATMIYYAERIGAQPNDPSASEHTHFKNIPIGFWWAVVTMTTLGYGDMYPQTWSGMLVGALCALAGVLTIAMPVPVIVNNFGMYYSLAMAKQ

SEQ ID NO: 12 - protein, 156 aa, Rattus norvegicus
PYYVTIFLTESNKSVLQFQNVRRVVQIFRIMRILRILKLARHSTGLQSLGFTLRRSYNELGLLILFLAMGIMIFSSLVFFAEKDEDDTKFKSIPASFWWATITMTTVGYGDIYPKTLLGKIVGGLCCIAGVLVIALPIPIIVNNFSEFYKEQKRQE

SEQ ID NO: 13 - protein, 149 aa, Homo sapiens
PYYIGLVMTDNEDVSGAFVTLRVFRVFRIFKFSRHSQGLRILGYTLKSCASELGFLLFSLTMAIIIFATVMFYAEKGSSASKFTSIPAAFWYTIVTMTTLGYGDMVPKTIAGKIFGSICSLSGVLVIALPVPVIVSNFSRIYHQNQRAD

(19) United States
(12) Patent Application Publication
(10) Pub. No.: US 2006/0167226 A1
(43) Pub. Date: Jul. 27, 2006

(54) Mutations of voltage-gated ion channnels that allow them to express a voltage-independent phenotype and an improved method to use the same

(75) Inventors: David Fedida, Vancouver (CA); David Steele, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Cardiome Pharma Corp., Vancouver (CA)

(21) Appl. No.: 10/520,780

(22) PCT Filed: Jul. 14, 2003
(86) PCT No.: PCT/US03/22165

§ 371 Date: Sep. 12, 2005

Related U.S. Application Data
(60) Provisional application No. 60/395,272, filed on Jul. 12, 2002.

(51) Int. Cl. C07K 14/705; C07H 21/04; C12P 21/06; C12N 15/74; C12N 1/18

(52) U.S. Cl. 530/350; 435/69.1; 435/320.1; 435/325; 536/23.5; 435/254.2

(57) ABSTRACT

The subject invention includes mutant voltage-gated ion channels that are open over a wide range of potential differences across membranes. The present invention also includes methods of use such mutant voltage-gated ion channels in cells with highly negative potential differences across their membranes. One preferred mutant voltage-gated ion channel is a channel with a mutation at the residue homologous to P513 in Kv1.5 and at least one mutation at one of the residues homologous to R400, R403, and R409 in Kv1.5.

### RELATED APPLICATIONS

This application claims the benefit of U.S. Provisional Application No. 60/395,272, filed Jul. 12, 2002, which is incorporated by reference herein in its entirety.

### FIELD OF THE INVENTION

This invention relates to mutated voltage-gated ion channels and their ability to complement cells deficient in uptake of the associated ion and to an improved method where the mutated ion channels function as a detection system for detecting inhibitors and/or activators of normally voltage-gated ion channels.

### BACKGROUND

Voltage-gated ion channels control the permeability of cell membranes to specific ions by opening and closing in response to changes in the potential difference across the membrane. Many voltage-gated ion channels play an essential role in information transfer and synaptic functions in neurons. Voltage-gated ion channels also participate in neuronal integration, cardiac pacemaking, muscle contraction, hormone secretion, cell volume regulation, lymphocyte differentiation, and cell proliferation. The ability to modulate the activity of voltage-gated ion channels, neuronal and otherwise, has implications in a wide range of physiological systems, diseases, and conditions including, without limitation, cardiac rhythm disturbances, diabetes, hypertension, asthma, and seizure disorders. As such, there is a need for methods of identifying compounds that can modulate the activity of voltage-gated ion channels. With current molecular biology techniques, voltage-gated ion channels may be expressed in a wide range of cells. The transgenic cells may be screened with libraries of compounds to identify compounds that modulate the activity. U.S. Pat. No. 5,795,770 discloses a method for screening yeast cells deficient in the uptake of potassium. However, most cells suitable for such screening have negative potential differences across the cell membranes that keep the voltage-gated ion channels closed. The problem is particularly acute with yeast, which would otherwise be a useful cell. The potential difference across the yeast cell membrane has been estimated at between −150 and −200 mV. The trk1 trk2 mutant disclosed in the U.S. Pat. No. 5,795,770 is even more hyperpolarized. Thus there is a need for mutations within conserved regions of voltage-gated ion channels that generate channels that are open at highly negative potential differences for screening in cells with highly negative potential differences across their cell membranes such as yeast.

Voltage-gated ion channels (potassium, sodium, and calcium channels) assemble as homotetramers (MacKinnon, 1991) and in some cases heterotetramers with each of the 4 subunits composed of 6 transmembrane helices. The S5 and 56 transmembrane helices in each subunit, and the interconnecting loops form the conduction pathway of the channels, also known as the pore ((MacKinnon, 1991; Doyle et al., 1998). The S1 through S3 helices do not have a dearly defined function yet, but likely are important determinants of channel structure and gating and thus are potential targets for mutations that alter gating (Seoh et al., 1996). The helices which are the preferred targets for mutations of the invention are the S4 helix, thought to be the voltage sensor (Noda et al., 1984; Papazian et al., 1991; Liman et al., 1991; Logothetis et al., 1992; Durell & Guy, 1992), and S6, which contributes to the pore of the channel.

The S4 helix is composed of a regular array of up to seven positively charged amino acids (arginine or lysine) with 2 intervening hydrophobic residues between each. The number, location, and character of these residues varies quite significantly in different channels and helps determine their different responses to changes in transmembrane voltage. As these charged residues are located within the transmembrane electric field, they should be influenced by changes in the voltage creating this field. The response of this positively charged helix to voltage changes is thought to lead to channel opening and this response can be measured electrophysiologically through gating currents and ionic currents while residue movement can also be directly measured using fluorescence. These measurements have led to many kinetic and biochemical models to explain the process of opening of ion channels ((Zagotta et al., 1994; Bezanilla et al., 1994; Schoppa & Sigworth, 1998; Baker et al., 1998).

Partly because the breadth of the transmembrane region where the S4 is located is unknown and the 7 charged S4 residues cover a large portion of the helix, it is thought that the residues will contribute varying amounts of charge to the total gating charge. This has been supported by mutational studies which look at total gating charge per channel following neutralization mutations of individual charged residues (Papazian et al., 1991; Perozo et al., 1994). In Shaker channels, neutralization of any one of the first 4 charged residues closest to the extracellular space (R362, R365, R368 and R371) shows significant reductions in total gating charge per channel. These 4 residues all seem to contribute to the total gating charge which moves upon depolarization. In Shaker channels there are an additional 3 charged residues (K374, R377 and K380) which are located closer to the intracellular space. Neutralization of K374 to glutamine results in no functional channel expression (Papazian et al., 1991; Perozo et al., 1994; Papazian et al., 1995; Aggarwal & MacKinnon, 1996); however, neutralization to a serine does permit expression and there is a reduction in total gating charge, though smaller than the reduction observed for the first 4 residues (Aggarwal & MacKinnon, 1996). Seoh et al. (Seoh et al., 1996) found no reduction in total gating charge when K374Q was paired with the mutation E293Q which restores expression. The 6th charged residue, R377 also shows no expression when mutated to the neutral glutamine (Papazian et al., 1995). However, histidine scanning mutagenesis suggests that this residue does not participate in gating (Bezanilla, 2000). Neutralization of the 7th residue, K380, does not lead to a reduction in gating charge (Papazian et al., 1991; Logothetis et al., 1992). It is difficult to draw any general conclusions for different channels regarding the effects of these neutralization mutations due to caveats regarding channel structure, but generally it seems that charged residues closer to the extracellular space (R362 through R371) contribute more to total gating charge than residues closer to the intracellular space (K374 through K380). Starkus, Rayner and associates ((Bao et al., 1999) have shown that the combined mutation of residues R362, R365 and R371 eliminates the voltage-sensitivity of channel gating in Shaker. Shaker channels harboring this combination have an opening probability of about 0.15 at all voltages between −160 and +80 mV.

Based on the crystal structure of the Streptomyces lividans KcsA channel ((Doyle et al., 1998), a tetramer of the N-terminal portion of the S6 transmembrane domain forms the inner mouth of the pore of potassium channels like Shaker and Kv1.5. Mutation of one residue in Shaker, P475, to alanine has been reported to prevent channel opening. Mutation of this residue in Shaker to aspartate or glutamate stabilizes the channel in an open state (Hackos and Swartz, Biophys. J. 78:398A, Abstract 2349; PCT/US01/03963). In the related potassium channel, hKv1.5, we have found as disclosed in this specification that neither combined mutation of the R362, R365 and R371 homologues nor mutation of the P475 are sufficient to rescue trk1 trk2 yeast, presumably owing to the hyperpolarized membrane.

### SUMMARY OF THE INVENTION

The present invention concerns modified voltage-gated ion channels that are open over a very wide potential difference range sufficient to allow screening in organisms such as yeast that have a highly negative potential difference across their membrane. The preferred embodiment of the present invention includes modification of a voltage-dependent ion channel in the S4 and S6 regions that allows the channel to be functional and pass ions effectively over a very wide potential range between −200 mV and +100 mV and beyond. Such modifications are most simply made by modification of a nucleic acid sequence encoding the channel and expressing the channel in a suitable cell type. A preferred embodiment is demonstrated in the Examples herein with the Kv1.5 channel, a voltage-gated potassium channel. The invention includes other voltage gated potassium, sodium and calcium ion channels as targets for screening in organisms with highly negative potential differences across their membrane. The preferred embodiment Kv1.5QPD may be replicated in these other voltage gated ion channels by mutation of homologous residues. Such channels would include, for example, all voltage-gated potassium channels (Kv), and others within the HGNC gene family such as hERG and KCNQ channels. In addition, the mutations are also relevant to channels that conduct other ions such as those within the Nav and Cav channels gene families. All these channels have readily definable S4 and S6 regions and identifiable homologous amino acid residues to those suggested here that embody the invention.

The present invention further provides a process for detecting modulators of voltage-dependent ion channels which comprises:

1. an improved method for treating modified cells with a test substance, wherein the modified cerevisiae cells express a nucleic acid sequence for the modified voltage-dependent ion channel, a preferred embodiment of which are yeast cells;

2. robust growth of modified Saccharomyces cerevisiae cells expressing the defined mutant channels(s) at very low concentrations of potassium ions (<0.5 mM K+), under which conditions untransformed trk1 trk2 yeast are unable to grow;

3. detecting any change in growth of the cells after treatment with the test substance.

One aspect of the present invention is potassium channel comprising a voltage-gated potassium channel which when expressed in a mutant yeast deficient in potassium uptake allows the mutant yeast to grow in the presence of media with very low potassium concentration, wherein said voltage-gated potassium channel comprises one or more mutations which produces a constitutively open voltage-gated potassium channel. In an embodiment of the present invention, the very low potassium concentration is about 2 mM or less, about 1 mM or less, about 0.7 mM or less, about 0.5 mM or less, or about 0.2 mM or less. In another embodiment of the invention, the mutant yeast lacks TRK1 or TRK1 and TRK2 potassium transporter activity. In still another embodiment, the mutations in the voltage-gated potassium channel are homologous to R400Q, and P513D in Kv1.5; are homologous to R403Q, and P513D in Kv1.5; are homologous to R409Q, and P513D in Kv1.5; are homologous to R400Q, R403Q, and P513D in Kv1.5; are homologous to R400Q, R409Q, and P513D in Kv1.5; are homologous to R403Q, R409Q, and P513D in Kv1.5; or are homologous to R400Q, R403Q, R409Q, and P513D in Kv1.5. In one embodiment, the voltage-gated potassium channel is a member of an ion channel family comprising Kv10, Kv11, Kv12, Kv1, Kv2, Kv3, or Kv4. In still another embodiment, the voltage-gated potassium channel is Kv1.5 or hERG.

Another aspect of the present invention includes a yeast cell comprising a deficiency in potassium uptake and a constitutively open voltage-gated potassium channel which allows said yeast cell to grow in the presence of media with very low potassium. In one embodiment, the deficiency is due to a lack of TRK1 or TRK1 and TRK 2 potassium transporter activity. In another embodiment, the very low potassium concentration is about 2 mM or less. In still another embodiment, the constitutively open voltage-gated potassium channel comprises two or more mutations that are homologous to R400Q, R403Q, R409Q, or P513D in Kv1.5. In yet another embodiment, the constitutively open voltage-gated potassium channel comprises mutations homologous to R400Q, and P513D in Kv1.5; homologous to R403Q, and P513D in Kv1.5; homologous to R409Q, and P513D in Kv1.5; homologous to R400Q, R403Q, and P513D in Kv1.5; homologous to R400Q, R409Q, and P513D in Kv1.5; homologous to R403Q, R409Q, and P513D in Kv1.5; or homologous to R400Q, R403Q, R409Q, and P513D in Kv1.5. In another embodiment, the constitutively open voltage-gated potassium channel is a member of an ion channel family comprising Kv10, Kv11, Kv12, Kv1, Kv2, Kv3, or Kv4. In an embodiment, the constitutively open voltage-gated potassium channel is Kv1.5 or hERG.

Another aspect of the present invention includes a recombinant nucleic acid molecule comprising a nucleic acid sequence encoding any of the above variations of the mutant voltage potassium channel disclosed above or in the specification. Another embodiment includes a promoter sequence operably linked such nucleic acid.

Still another aspect of the present invention includes methods comprising providing a cell deficient in potassium uptake expressing the potassium channels disclosed above, wherein said cell has a high negative potential across the plasma membrane; growing the cell in very low potassium; adding a compound; and assaying the effect of the compound on the growth of the cell. In one embodiment, the cell is a yeast cell. In another embodiment, the yeast cell lacks TRK1 or TRK1 and TRK2 transporter activity. In still another embodiment, the yeast cell is S. cerevisiea. In yet anther embodiment, the effect of the compound on the growth of the cell represents a procedure to determine the modulating activity of said compound on the potassium channel. In certain embodiments, the modulating activity refers to the inhibition activity of said compound on the potassium channel.

### BRIEF DESCRIPTION OF THE FIGURES

FIG. 1. Neither the Kv1.5 triple S4 mutant (A) nor the Kv1.5 P513D (B) S6 mutant grows on low K+ media. Each row represents different transformants in three columns of yeast dilutions, 1×10⁻⁴, 1×10⁻⁵ and 1×10⁻⁶/ml. The control was vector only, without channel in rows 1, 3 and 5. The channel transformants are in rows 2, 4 and 6, labeled Q124- in (A) and P513D in (B). Only row 4 of panel B shows apparent growth, but this growth proved to result from pseudorevertant activity. The same is true of the papillae seen in some patches.

FIG. 2. Growth on agar plates of yeast strains containing the Kv1.5 S6, triple S4 mutant in low potassium media. Six of nine transformants grew well on 7 mM K medium. Two of nine control transformants grew weakly. Control was vector only, without channel in. Above are three examples. Top and bottom are channels, showing good growth and middle set of plaques are vector alone. Three columns of yeast dilutions, 1×10⁻⁴, 1×10⁻⁵ and 1×10⁻⁶/ml. Heaviest growth seen at 1×10⁻⁴ yeast/ml (right column). The left column only shows one colony in the top row, and is invisible in the lower two rows.

FIG. 3. A graph of the actions of various Kv1.5 antagonists on the growth of Kv1.5 S6, triple 54 mutants in low potassium media. Yeast growth response for 6 Kv1.5 antagonists of differing potencies. The rank order for inhibition of yeast growth is from Compound 1 to Compound 6. This order is very close to that obtained from the patch clamp assay. Individual data points are shown for two separate assays, and mean data as the solid line.

FIG. 4. Alignment of select voltage-gated ion channels.

FIG. 5. Comparison of (A) hERG D540 vs. Kv1.5QPD and (B) hERG D540 vs WΔ3. (A) shows growth on 100 mM K+ (left plate) and growth on 0.5 mM KB (right plate). Kv1.5QPD (left side of each plate) allows growth at both potassium concentrations. hERG D540 (right side of each plate) does not support growth at 0.5 mM K+. (B) shows that hERG D540 (right side of plate) allows growth on 5 mM K+ while WΔ3 alone (left side of plate) cannot grow on 5 mM K+.

### BRIEF DESCRIPTION OF THE TABLES

Table 1. Inhibition of growth in the presence of the specific potassium channel-inhibiting agent, 4-aminopyridine.

### DETAILED DESCRIPTION OF THE INVENTION

Definitions

The following definitions apply to terms used throughout this specification unless otherwise limited in specific instances.

The term ‘modified’ as used with respect to a cell, refers to a cell in which the wild-type genome has been altered by the addition of one or more heterologous genes, a deficiency in one or more wild-type genes, or a combination thereof. Such modifications may be carried out by transformation and homologous recombination through techniques well understood by those having ordinary skill in the art.

The term ‘functional derivative or mutant thereof’ as used with respect to a protein refers to a protein differing from the subject protein by one or more amino acid residues but still having the ion channel transport function of the protein and greater than about 90% sequence homology. Such derivatives and mutants may include proteins that differ from the wild-type protein by amino acid substitutions, deletions, disruptions, and the like. Such differences may be accomplished by genetic means, using such techniques as site-directed mutagenesis, or mutagenic PCR prior to translation, or by chemical means using proteases and/or ligases after translation.

The functional derivatives of human or mammalian ion channel genes incorporate specific mutations into the voltage-sensing regions of the S4 transmembrane domain and in the pore-lining surface of S6 in each instance. Specifically three of the outer positively charged arginine or lysine residues of the S4 domain are mutated to glutamine residues and a conserved proline in the S6 domain is mutated to aspartate. This alters the functional properties of channel opening, also known as gating. Potassium ion channels normally only gate open when the perceived potential across the protein (usually incorporated into a lipid bilayer) exceeds, or becomes positive to −40 mV on the inside. With the present modification the channels gate across an extremely wide potential range exceeding −200 mV in the negative direction and +100 mV in the positive direction. This allows them to be open at transmembrane potentials at which they are normally dosed (i.e. negative to −60 mV on the inside).

The term “channel ion”, as used herein, refers to the ion which a given voltage-gated ion channel selectively passes when open. The voltage-gated ion channels of the present invention may be potassium, sodium, or calcium channels.

As used herein, the term “wide-potential-range ion channel” refers to a mutant voltage-gated ion channel that is open to a sufficient degree at highly negative potential differences to complement a yeast deficient in uptake of the channel ion. A preferred embodiment of wide-potential-range ion channels is one that can allow the yeast deficient in uptake of the channel ion when the channel ion is supplied at a low concentration in the media. Thus a wide-potential-range ion channel may allow yeast deficient in uptake of the channel ion when the channel ion is less than 2 mM, less than 1.0 mM, less than 0.5 mM or less than 0.2 mM. The Examples provide a preferred embodiment of a wide-range-potential ion channel in the form of Kv1.5QPD.

As used herein, the term “highly-negative potential difference” means a potential difference across a membrane of −200 mV or greater. In some embodiments, the highly-negative potential difference is −225 mV or greater, −250 mV or greater, or −300 mV or greater. In a preferred embodiment, the potential difference is that found in trk1 trk2 yeast grown in media with 0.5 mM potassium.

Voltage-Gated Ion Channels

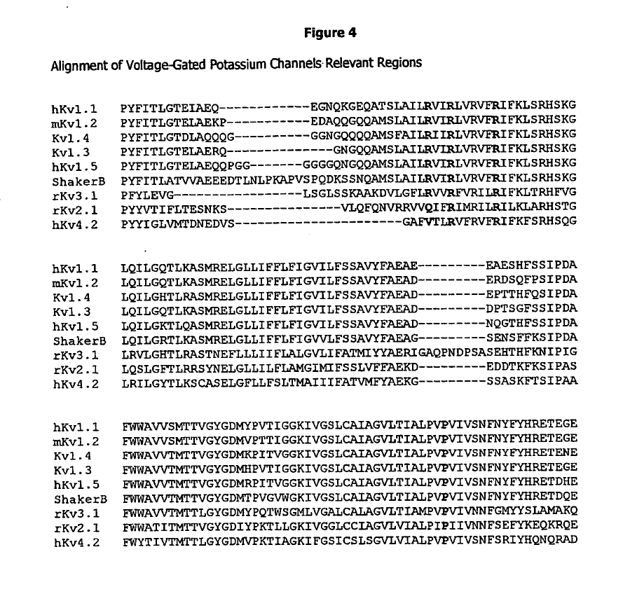

Voltage-gated ion channels that are mutated in the present invention are from related families of proteins. Voltage-gated ion channels may be readily identified by function, by structure (both secondary and tertiary), and by sequence homology (primary structure). A hallmark of the voltage-gated ion channels are the six putative transmembrane spanning helices S1-6 and the “PVP” motif (which is not invariant, e.g., rKv2.1 has a PIP sequence). Within the larger super-family are various families including potassium gated (Kv), sodium gated (Nav), and calcium gated (Cav). The voltage-gated potassium channels fall into a super-family that uses the nomenclature Kv. One family includes four sub-families that were originally named for the four related voltage gated potassium channels from Drosophila: Shaker (Kv1); Shab (Kv2); Shaw (Kv3); and Shal (Kv4). Shaker and Shal are characterized as having rapid current activation and inactivation, while Shab and Shaw are delayed rectifier channels that are characterized as having slow inactivation and non-inactivation. Homologues in each subfamily have been identified in humans, rodents, and other mammals. FIG. 4 shows an alignment of representative members of the Kv1-K4 sub-families.

In addition, the Kv super-family has a number of other families. Kv10-Kv12 make up the eagrelated family. Human erg (Herg or Kv11.1) is an important member of this family. The Herg gene corresponds to the LQT-2 genetic locus. Long QT (LQT) syndromes are inherited cardiac disorders characterized by prolonged QT interval on the electrocardiogram (ECG) associated with syncopal attacks, and high risk of sudden death due to ventricular tachyarrhythmia (Schwartz, 1985). Although congenital LQTs are not frequent diagnoses, acquired forms of abnormal repolarization and susceptibility to arrhythmia are very common. Occasional syncopes (loss of consciousness) are due to malignant tacharyrhythmias, usually torsade de pointes. Sudden death may occur by transformation of these ventricular arrhythmias into ventricular fibrillation. Mutations in Herg are known to cause autosomal dominant LQT syndrome. Herg ion channels are inwardly rectifying potassium channels that have voltage gating properties similar to other members of other eag-related channels. The most common form of LQTs, however, is due to mutations in the KvLQT1 (Kv7.1) potassium channel gene that encodes an outwardly rectifying, voltage-gated K+ channel with six transmembrane domains.

The present invention provides mutant voltage-gated ion channels that remain open at extremely negative potential differences. The mutant voltage-gated ion channels of the present invention are characterized by their ability to complement yeast deficient in potassium uptake even when grown at very low potassium levels. The preferred levels are 0.5 mM K⁺ or lower. A preferred embodiment of the present invention is a channel that includes mutations at residues homologous to R400, R403, R409, and/or P513 in Kv1.5. A further preferred embodiment of the present invention is a channel that includes mutations homologous to R400Q, R403Q, R409Q, and/or P513D in Kv1.5.

The mutant voltage-gated ion channels of the present invention may be generated by a wide range of well known molecular biology techniques. Detailed protocols for numerous such procedures are described in, e.g., in Ausubel et al. Current Protocols in Molecular Biology (Supplemented through 2000) John Wiley & Sons, New York; Sambrook et al. Molecular Cloning—A Laboratory Manual (2^{nd} Ed.), Vol. 1-3, Cold Spring Harbor Laboratory, Cold Spring Harbor, N.Y., 1989, and Berger and Kimmel Guide to Molecular Cloning Techniques, Methods in Enzymology volume 152 Academic Press, Inc., San Diego, Calif.

Transgenic Cells of the Present Invention

The present invention also encompasses transgenic cells expressing the mutant voltage-gated ion channels. A preferred cell is one with highly-negative potential differences across its cell membrane, such as yeast. One of ordinary skill in the art will be able to generate the transgenic cells of the present invention using routine methods of molecular biology. The transgene may be constructed within or introduced into an expression vector appropriate for the cell of interest. Many different expression vectors exist for expression in a variety of prokaryotic and eukaryotic cells. For ease of manipulation, a shuttle vector may be used to allow manipulation of the construct in a prokaryotic cell and later expression in a eukaryotic cell.

Such expression vectors typically contain a promoter and a transcription termination element. The transgene should be operably linked to a promoter that functions in the cell of interest. The promoter may be constitutive or inducible. The choice of promoter will be dictated by the particular application. One of skill in the art would have no difficulty in selecting the appropriate promoter. Optionally, the expression vector may also contain other useful elements such as ribosome binding sites, polyadenylation signals, etc. as appropriate.

Once the expression vector has been constructed, it may be introduced into the cell of interest by a number of standard transfection methods. These may include calcium phosphate transfection, protoplast fusion, electroporation, microprojectile bombardment, liposomes, microinjection, viral vectors and any other known method for introducing cloned nucleic acid sequences into the cell of interest as is appropriate. All that matters is that the nucleic acid is introduced into the cell of interest. The expression vector may integrate into the genetic material of the host cell, may replicate as an independent genetic element, or may remain in the cell transiently.

Once the expression vector has been introduced into the cell. The cell may be cultured under conditions that allow expression of the mutant voltage-gated ion channel when desired.

Screening Methods of the Present Invention

The present invention also provides methods for screening compounds for their ability to modulate the activity of voltage-gated ion channels. The mutant voltage-gated ion channels of the present invention may be expressed in a cell with a highly negative potential difference across the membrane. The cell should be deficient in uptake of the channel ion of the voltage-gated ion channel of interest such that growth is inhibited or hindered on media containing low concentrations of the channel ion of the mutant voltage-gate ion channel. Such a cell-based system may be assayed to determine whether a given compound modulates the activity of the voltage-gated ion channel. The modulation may by detected by the effect on the growth of the cell or other methods that detect the presence of the channel ion within the cell such as use of compounds that fluoresce in the presence of the channel ion.

Cells deficient in the uptake of the channel ion of the voltage-gated ion channel of interest may be generated by many different techniques available to those of skill in the art. The cell may be made deficient by targeted knock-out of genes involved in conveying the channel ion across the membrane. One of skill in the art will recognize that this may be accomplished in several ways, including knock-out of the genes for the proteins that actually convey the channel ions across the membrane as channels or transporters; knock-out of the genes that activate the transcription of such genes; etc. In addition, the cell may be made by screening for naturally occurring mutations or mutations induced by a variety of methods such as chemical mutagenesis, transposon insertion, radiation, etc. Furthermore, the cell may be made deficient by addition of compounds that inhibit the cell's natural mechanisms for uptake of the channel ion. The method used to make the cell deficient is not important, so long as the cell is rendered deficient such that the cell cannot grow in media with low concentrations of the channel ion.

The compounds tested for the ability to modulate the activity of voltage-gated ion channels can include any small chemical entity as well as biological entities such as proteins, sugars, nucleic acids, lipids, and any combination thereof. It will be appreciated that the screening method of the present invention may be applied to large numbers of compounds. In a preferred embodiment, the assay is part of an automated high-throughput screening apparatus designed to screen large libraries of chemical and/or biological entities.

The compounds identified may be used to modulate the activity of the voltage-gated ion channels in vivo by administering the compound to an animal that expresses the voltage-gated ion channel. It will be appreciated by those of ordinary skill in the art that such compounds will be defined by their function in the screening assays of the present invention as well as structurally for those that modulate by direct interaction with the voltage-gate ion channel. The structure of such a compound must at least in part be defined by the structure of the voltage-gated ion channel much as the structure of an electrical plug is in part defined by an electrical socket. Thus, the compounds identified in the assays of the present invention and their methods of use to modulate the voltage-gated ion channels in animals is an aspect of the present invention.

A preferred embodiment of the present invention is yeast cell deficient in potassium uptake for use in screening voltage-gated potassium channel. The yeast cell of the present invention possesses deficient alleles in the TRK1 and TRK2 genes required for potassium uptake. The modified alleles are genetically stable and recessive, so they can be complemented with activities encoded by heterologous genes introduced into the strain (U.S. Pat. No. 5,795,770). Pseudorevertants do arise with high frequency, however, and can complicate the strain's usefulness in liquid culture. The present invention provides a method to overcome this problem in liquid culture.

A variety of the modified voltage-gated ion channels containing the combined mutations in homologous positions to those described above may be introduced into this yeast strain to assess whether these channels can complement the growth defect on potassium-deficient media. The application results in a yeast strain expressing a normally voltage-gated, modified foreign ion channel, useful in a screen for modulators of the channels.

A yeast strain expressing an altered voltage-gated ion channel can be adapted to natural products screening. A simple screen design involving growth inhibition or ion uptake on solid plates or in liquid culture may detect compounds that modulate channel function. The screen may involve such modifications as sensitivity to the pH of the media, growth in different concentrations of oxygen and carbon dioxide, changes in growth on different concentrations of potassium in the medium, or changes in ion uptake.

### EXAMPLES

### Example 1

### Construction of Mutant Kv1.5 Channels

The gene for human potassium channel Kv1.5 was cloned into yeast expression vectors such that expression was under the control of the GALL or PGK promoter. Site-directed mutagenesis was then used to recode the Kv1.5 gene such that amino acids R400, R403, and R409 would be replaced by glutamine and amino acid P513 would be replaced by aspartate in the expressed channel. The coding region of the human gene for Kv1.5 was subcloned into pYC2 (Invitrogen) as an approximately 2 kb HindIII-NotI fragment. Site-directed mutagenesis was conducted in two steps using the Quick Change Mutagenesis Kit (Invitrogen). The oligonucleotides 5′-ATCCTCCAAGTCATCCAACTGGTCCGGGTGTTCCAAATCTTCAAG-3′ (SEQ ID NO: 1) and 5′-TTGMGATTGGAACACCCGGACCAGTrGGATGACrGGAGGATG-3′(SEQ ID NO: 2) encoded the five nucleotide changes in the S4-coding region from wild-type. The oligonucleotides 5′-ATTGCCCTGCCTGTGGACGTCATCGTCTCCAAC-3′ (SEQ ID NO: 3) and 5′-TTGGAGACGATGACGTCCACAGGCAGGGCAATG-3′ (SEQ ID NO: 4) encoded the 2 nucleotide changes in the S6 coding region. The presence of the predicted nucleotide changes was confirmed by DNA sequencing. Further sequencing confirmed the absence of additional mutations.

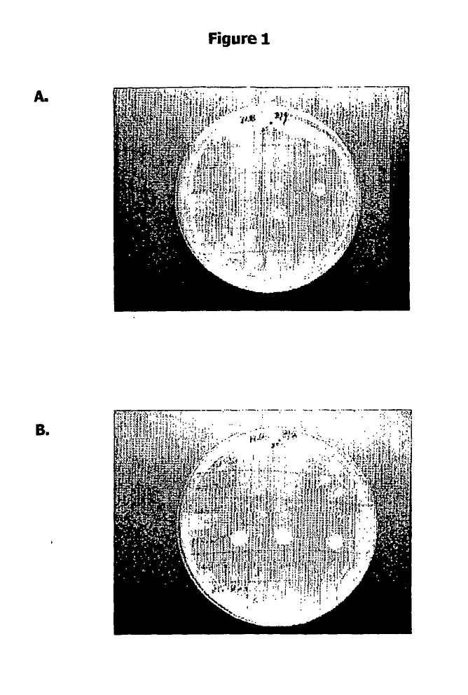

The replacement of the three arginines was based on the fact that in a related channel, Shaker, the analogous replacements yield a channel which gates in a voltage-insensitive manner ((Bao et al., 1999). The proline at position 513 was replaced on the basis of a report that an analogous mutation in Shaker yielded a constitutively open channel (Hackos and Swartz, Biophys. J. 78:398A, Abstract 2349). In Kv1.5, neither the changes in S4 nor those in S6 proved sufficient on their own to allow yeast growth in the screen described below (FIG. 1).

The plasmid containing the mutant Kv1.5 channel (Kv1.5m) of the present invention was transformed into the trk1trk2 yeast strain using a Lithium acetate procedure. Unlike the trk1 trk2 yeast transformed with the empty vector, the quadruple-mutant Kv1.5m construct expressed from the GAL1 promoter (with galactose used as the carbon source) when transformed into these yeast, was found to allow growth on media containing 5 mM potassium (growth on glucose, as expected, required 100 mM potassium. Kv1.5m is under the control of the GAL1 promoter, and, thus, should be expressed only when galactose is the sole carbon source). A similar phenomenon was observed in yeast transformed with a vector expressing Kv1.5m from the constitutive PGK promoter, but independent of carbon source. Neither the combined three arginine mutations nor the proline mutation on their own conferred growth capability on low potassium to the yeast (see above, FIG. 1). The Kv1.5m-transformed strain is a preferred embodiment of this invention.

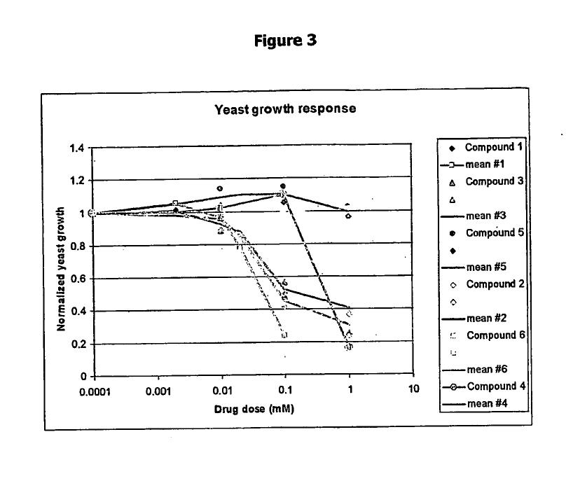

Growth of the trk1 trk2 strain expressing Kv1.5m on low potassium could be blocked by the addition of the Kv1.5-blocking 4-aminopyridine (Table 1), as well as by other agents known to block Kv1.5 (FIG. 3). Growth of the trk1trk2 strain transformed with a similar construct expressing the inward rectifier KIR2.1 rather than Kv1.5m was not inhibited by these drugs.

The trk1 trk2 mutant yeast, WΔ3 (MATa, leu2-3,112, trp1-1, ura3-3, ade2-1, his3-1, can1-100, trk1::LEU2, trk2::HIS3), was a gift of Alonso Rodriguez-Navarro, Escuela Tecnica Superior de Ingenieros Agronomos, Madrid, Spain.

### Example 2

### Introduction of the Ion Channel Mutant Forms into the Yeast

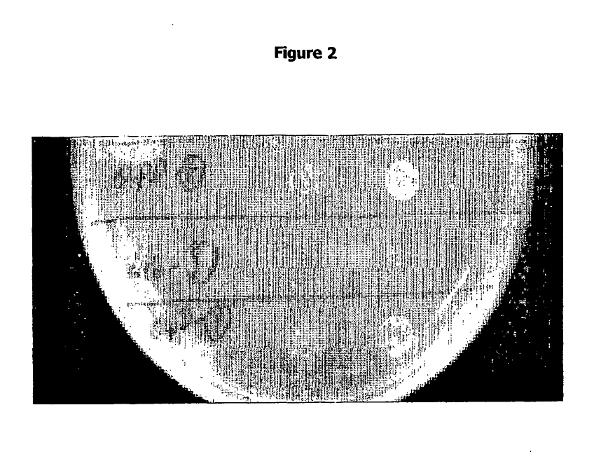

YPD, YNB and low-salt (LS) media were prepared by standard methods (Sherman, Fink and Hicks, Methods in Yeast Genetics, Cold Spring Harbor, 1986; Rodriguez-Navarro and Romos, J. Bacteriol. 159:940-945,1984; Wickerham, L. J., U.S. Dept of Agriculture Technical Bulletin No. 1029, 1951). Introduction of the modified gene incorporated in expression vectors into yeast was by a simple transformation procedure. Yeast cells were incubated overnight at room temperature with the plasmid DNA and carrier (salmon sperm DNA) in 100 mM Lithium acetate, 10 mM Tris HCl, 1 mM EDTA, 34% polyethylene glycol (m.w. 4000). Initial selection and subsequent screening of transformants was carried out on media lacking uracil (YNB-uracil/100 mM KCl) to maintain selection for the plasmids. 100 mM KCl was included to allow time for Kv1.5m expression. The parent strain, WΔ3, is capable of growth on 100 mM KCl but not on 7 mM KCl or lower. Transformant colonies were then transferred to low-potassium (7 mM) media lacking uracil (YNB-uracil) to verify functional expression of the channel as shown in FIG. 2.

### Example 3

### Function of the Cells as Reporters in Drug Screening

Expression of the cloned inserts is under the control of either the constitutively active PGK promoter or the inducible GAL1 promoter. The latter requires growth in/on media containing galactose as sole carbon source. The screening method involves adding the compound to be screened to the growth media of trk1 trk2 yeast expressing the modified potassium channel and determining whether the yeast's growth is inhibited. The tested compound may be incorporated in liquid or solid growth media or added ectopically to solid growth media.

A major problem in using trk1 trk2 yeast is the high number of pseudorevertants that generally arise on low potassium media (Liang et al., 1998). This is not a significant problem on solid media, since the pseudorevertants appear as individual colonies on a non-growing background (Graves & Tinker, 2000). But in liquid cultures, the frequency is high enough that virtually all overnight cultures are overtaken by the pseudorevertants. This problem is endemic in cultures grown in 7 mM potassium as described by all other workers. Since insensitive pseudorevertants mask any block of the expressed channel, drug sensitivity testing in liquid culture is nearly impossible under these conditions

In the present invention a surprisingly simple method was found by the inventors to overcome this problem. Our yeast strain, when expressing the mutant Kv1.5 channel, is capable of consistent growth on as little as 0.5 mM potassium. Very few pseudorevertants are capable of growth at this potassium concentration; the vast majority of liquid cultures of the parent strain show absolutely no growth even after more than 24 hours culture. When expressing the mutant Kv1.5 channel, growth is robust and can be blocked by known Kv1.5 channel blockers.

Detailed Description of the Methods Required for a Liquid Culture Media Assay of Kv1.5:

Low salt media was made according to Wickerham, L. J. (1951) (Taxonomy of Yeasts. United States Department of Agriculture Technical Bulletin No. 1029), except that the final potassium phosphate concentration was 0.5 mM.

1. Streak out WΔ3 and Kv1.5m transformed WΔ3 (“Kv1.5m”) culture from frozen stock onto YPD+100 mM K+ and YNB+100 mM K+, ura-agar media plates, respectively, for fresh colonies.

2. The day before doing the drug assay, use the fresh colonies to grow overnight cultures of WΔ3 in liquid YPD+100 mM K+ media, and grow “Kv1.5 m” in liquid YNB+100 mM K+, ura-media.

3. Collect the overnight yeast cells and wash them in sterile distilled water. Resuspend WΔ3 cells in the same volume of LS+100 mM K++ uracil media, and resuspend “Kv1.5 m” cells in the same volume of LS+0.5 mM K+, ura-media. These are the seed cultures for the drug assay.

4. In Falcon 2059 tubes, prepare various concentrations of drugs in LS+0.5 mM K+, ura-media or LS+100 mM K++ uracil media, and also set up solvent controls for each drug concentrations used. Here is an example:

Drug X stock solution (10 mM) is dissoved in 0.5% Tween-20. Final concentrations that we test are 1 mM, 0.1 mM, and 0.01 mM.

| WΔ3 control set up: | 0.1 mM | | 0.1 mM | | 0.01 mM | | 0 mM |
|---|---|---|---|---|---|---|---|
| drug × (10 mM) | 0.5 | ml | 0.05 | ml | 0.005 | ml | — |
| LS + 100 mM K+ + | 4.5 | ml | 4.95 | ml | 4.995 | ml | 5 ml |
| uracil media | | | | | | | |

Then 25 micro liter of the seed culture of WΔ3 from step 3 is inoculated into each tube.

| Solvent control set | | | | | | | |
|---|---|---|---|---|---|---|---|
| up: | 0.1 mM | | 0.1 mM | | 0.01 mM | | 0 mM |
| 0.5% Tween-20 | 0.5 | ml | 0.05 | ml | 0.005 | ml | — |
| LS + 100 mM K+ + | 4.5 | ml | 4.95 | ml | 4.995 | ml | 5 ml |
| uracil media | | | | | | | |

Then 25 micro liter of the seed culture of WΔ3 from step 3 is inoculated into each tube.

| Drug assay set up: | 0.1 mM | | 0.1 mM | | 0.01 mM | | 0 mM |
|---|---|---|---|---|---|---|---|
| Drug × (10 mM) | 0.5 | ml | 0.05 | ml | 0.005 | ml | — |
| LS + 0.5 mM K+, | 4.5 | ml | 4.95 | ml | 4.995 | ml | 5 ml |
| ura-media | | | | | | | |

Then 250 micro liter of the seed culture of “Kv1.5m” from step 3 is inoculated into each tube.

5. Measure the starting OD600 by taking out 0.1 ml of the sample from each tube and diluting lox for measuring. Grow cultures at 300 C shaker at 300 rpm.

6. Get 0.1 ml of samples from each tube and measure their OD600 at 2 hr, 4 hr, 6 hr, 8 hr, and 24 hr.

Table 1 shows that the standard potassium channel blocker, 4-aminopyridine (4-AP) can block the Kv1.5m channels in the liquid media assay:

Inhibition of the Growth of QPD WΔ3
Transformants by 4-AP in Liquid Culture

| Media | OD600 (Batch A) | OD600 (Batch B) |
|---|---|---|
| LS + 0.5 mM K⁺ | 2.33 | 2.33 |
| LS + 0.5 mM K⁺ + 0.1 mM 4-AP | 2.55 | 2.5 |
| LS + 0.5 mM K⁺ + 1 mM 4-AP | 1.11 | 1.11 |

The reduction of 50% at 1 mM 4-AP in both batches at OD600 indicates an inhibition of yeast growth by the presence of the compound.

In addition, this liquid assay reproducibly matches the rank order of potency of a number of Kv1.5 antagonists, as shown in FIG. 3. The potency (EC50) of the various compounds used on Kv1.5 as determined by patch clamp metholodogy (n=3) determinations, was, in rank order of potency:

Compound 1 (Stock solution dissolved in 0.5% Tween 20 at 10 mM, EC50=0.29-0.43 micro molar)

Compound 2 (water soluble, EC50=0.4 micro molar)

Compound 3 (water soluble, EC50=3.6 micro molar)

Compound 4 (water soluble, EC50=5.4 micro molar)

Compound 5 (water soluble. EC50=6.1-10.2 micro molar)

Compound 6 (water soluble, EC50=28 micro molar)

### Example 4

### Test with Another Shaker Channel

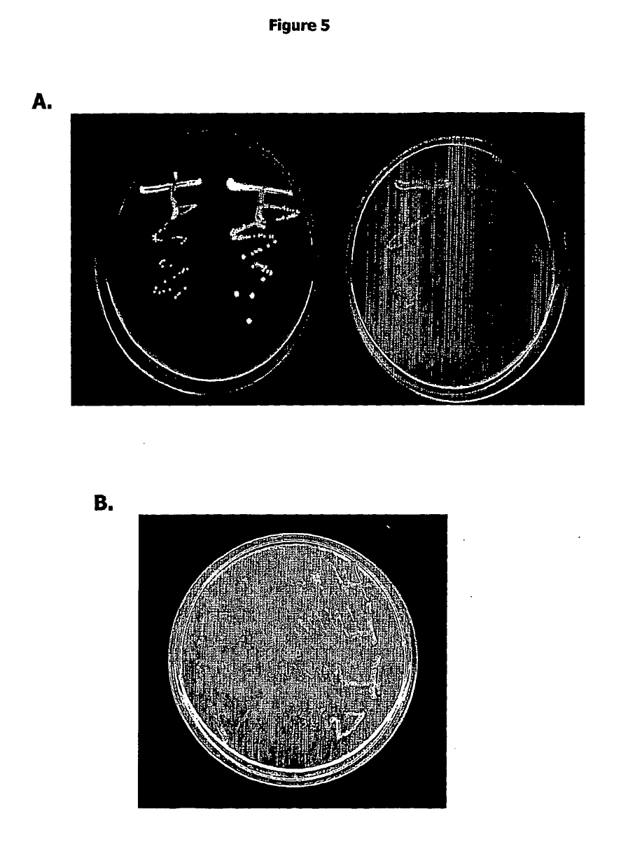

A second shaker channel was tested to determine whether a previously reported single mutation would function to allow a trk1 trk2 yeast to grow at very low potassium concentration. hERG with a single mutation, D540K, has been reported to yield a channel that is open at hyperpolarized potentials (Sanguinetti and Xu, 1999). These workers reported that the D540K mutation (in the channel's S4-S5 linker) causes hERG to open at hyperpolarized potentials into a novel long-lived open state. This mutant may be used for screening at 5.0 mM K+. As shown in FIG. 5, however, this mutation is not sufficient to allow trk1 trk2 yeast growth on 0.5 mM K+.

One of skill in the art will recognize, or be able to ascertain using no more than routine experimentation, many equivalents to the specific embodiments of the invention described herein. Such equivalents are intended to be encompassed by the present invention.

### REFERENCES

All references cited hereunder are hereby incorporated by reference.
Aggarwal, S. K. & MacKinnon, R. (1996). Contribution of the S4 segment to gating charge in the Shaker K+ channel. Neuron 16, 1169-1177.
Baker, O. S., Larrson, R., Mannuzzu, L. M., & Isacoff, E. Y. (1998). Three transmembrane conformations and sequence-dependent displacement of the S4 domain in Shaker K+ channel gating. Neuron 20, 1283-1294.
Bao, H. X., Hakeem, A., Henteleff, M., Starkus, J. G., & Rayner, M. D. (1999). Voltage-insensitive gating after charge-neutralizing mutations in the S4 segment of Shaker channels. Journal of General Physiology 113, 139-151.
Bezanilla, F. (2000). The voltage sensor in voltage-dependent ion channels. Physiological Reviews 80, 555-592.
Bezanilla, F., Perozo, E., & Stefani, E. (1994). Gating of Shaker K+ channels: II. The components of gating currents and a model of channel activation. Biophysical Journal 66, 1011-1021.
Doyle, D. A., Cabral, J. M., Pfuetzner, R. A., Kuo, A. L., Gulbis, 3. M., Cohen, S. L., Chait, B. T., & MacKinnon, R. (1998). The structure of the potassium channel: Molecular basis of K+ conduction and selectivity. Science 280, 69-77.
Durell, S. R. & Guy, H. R. (1992). Atomic scale structure and functional models of voltage-gated potassium channels. Biophysical Journal 62, 238-250.
Graves, F. M. & Tinker, A. (2000). Functional expression of the pore forming subunit of the ATP-sensitive potassium channel in Saccharomyces cerevisiae. Biochemical and Biophysical Research Communications 272, 403-409.
Liang, H., Ko, C. H., Herman, T., & Gaber, R. F. (1998). Trinucleotide insertions, deletions, and point mutations in glucose transporters confer K+ uptake in Saccharomyces cerevisiae. Mol. Cell. Biol. 18, 926-935.
Liman, E. R., Hess, P., Weaver, F., & Koren, G. (1991). Voltage-sensing residues in the 54 region of a mammalian K+ channel. Nature 353, 752-756.
Logothetis, D. E., Movahedi, S., Satler, C., Lindpaintner, K., & Nadal-Ginard, B. (1992). Incremental reductions of positive charge within the S4 region of a voltage-gated K+ channel result in corresponding decreases in gating charge. Neuron 8, 531-540.
MacKinnon, R. (1991). New Insights into the structure and function of potassium channels. Curr. Opin. Neurobiol. 1, 14-19.
Noda, M., Shimizu, S., Tanabe, T., Takai, T., Kayano, T., Ideda, T., Takahashi, H., Nakayama, H., Kanaoka, Y., Minamino, N., & et al. (1984). Primary structure of Electrophorus electricus sodium channel deduced from cDNA sequence. Nature 312, 121-127.
Papazian, D. M., Shao, X. M., Seoh, S.-A., Mock, A. F., Huang, Y., & Wainstock, D. H. (1995). Electrostatic interactions of S4 voltage sensor in Shaker K+ channel. Neuron 14, 1293-1301.
Papazian, D. M., Timpe, L. C., Jan, Y. N., &Jan, L. Y. (1991). Alteration of voltage-dependence of Shaker potassium channel by mutations in the S4 sequence. Nature 349, 305-310.
Perozo, E., Santacruz-Toloza, L., Stefani, E., Bezanilla, F., & Papazian, D. M. (1994). S4 mutations alter gating currents of Shaker K channels. Biophysical Journal 66, 345-354.
Sanguinetti, M. C. and Xu, Q. P. (1999) Mutations of the S4-S5 linker alter activation properties of HERG potassium channels expressed in Xenopus oocytes. J. Physiol. 514:667-675.
Schoppa, N. E. & Sigworth, F. J. (1998). Activation of Shaker potassium channels III. An activation gating model for wild-type and V2 mutant channels. Journal of General Physiology 111, 313-342.
Seoh, S. A., Sigg, D., Papazian, D. M., & Bezanilla, F. (1996). Voltage-sedsing residues in the S2 and S4 segments of the Shaker K+ channel. Neuron 16, 1159-1167.
Zagotta, W. N., Hoshi, T., & Aldrich, R. W. (1994). Shaker potassium channel gating. III: Evaluation of kinetic models for activation. Journal of General Physiology 103, 321-362.

We claim:

1. A potassium channel comprising a voltage-gated potassium channel which when expressed in a mutant yeast deficient in potassium uptake allows the mutant yeast to grow in the presence of media with very low potassium concentration, wherein said voltage-gated potassium channel comprises one or more mutations which produces a constitutively open voltage-gated potassium channel.

2. The potassium channel of claim 1, wherein the very low potassium concentration is about 2 mM or less.

3. The potassium channel of claim 1, wherein the very low potassium concentration is about 1 mM or less.

4. The potassium channel of claim 1, wherein the very low potassium concentration is about 0.7 mM or less.

5. The potassium channel of claim 1, wherein the very low potassium concentration is about 0.5 mM or less.

6. The potassium channel of claim 1, wherein the very low potassium concentration is about 0.2 mM or less.

7. The potassium channel of claim 1, wherein the mutant yeast lacks TRK1 or TRK1 and TRK2 potassium transporter activity.

8. The potassium channel of claim 7, wherein the mutant yeast lacks TRK1 and TRK2 potassium transporter activity.

9. The potassium channel of claim 1, wherein the mutations in the voltage-gated potassium channel are homologous to R400Q, and P513D in Kv1.5.

10. The potassium channel of claim 1, wherein the mutations in the voltage-gated potassium channel are homologous to R403Q, and P513D in Kv1.5.

11. The potassium channel of claim 1, wherein the mutations in the voltage-gated potassium channel are homologous to R409Q, and P513D in Kv1.5.

12. The potassium channel of claim 1, wherein the mutations in the voltage-gated potassium channel are homologous to R400Q, R403Q, and P513D in Kv1.5.

13. The potassium channel of claim 1, wherein the mutations in the voltage-gated potassium channel are homologous to R400Q, R409Q, and P513D in Kv1.5.

14. The potassium channel of claim 1, wherein the mutations in the voltage-gated potassium channel are homologous to R403Q, R409Q, and P513D in Kv1.5.

15. The potassium channel of claim 1, wherein the mutations in the voltage-gated potassium channel are homologous to R400Q, R403Q, R409Q, and P513D in Kv1.5.

16. The potassium channel of claim 1 or 7, wherein the voltage-gated potassium channel is a member of an ion channel family comprising Kv10, Kv11, and Kv12.

17. The potassium channel of claim 1 or 7, wherein the voltage-gated potassium channel is a member of the Kv10 ion channel family.

18. The potassium channel of claim 1 or 7, wherein the voltage-gated potassium channel is a member of the Kv11 ion channel family.

19. The potassium channel of claim 1 or 7, wherein the voltage-gated potassium channel is a member of the Kv12 ion channel family.

20. The potassium channel of claim 1 or 7, wherein the voltage-gated potassium channel is a member of an ion channel family comprising Kv1, Kv2, Kv3, and Kv4.

21. The potassium channel of claim 1 or 7, wherein the voltage-gated potassium channel is a member of the Kv1 ion channel family.

22. The potassium channel of claim 1 or 7, wherein the voltage-gated potassium channel is a member of the Kv2 ion channel family.

23. The potassium channel of claim 1 or 7, wherein the voltage-gated potassium channel is a member of the Kv3 ion channel family.

24. The potassium channel of claim 1 or 7, wherein the voltage-gated potassium channel is a member of the Kv4 ion channel family.

25. The potassium channel of claim 1 or 7, wherein the voltage-gated potassium channel is Kv1.5 or hERG.

26. The potassium channel of claim 1 or 7, wherein the voltage-gated potassium channel is Kv1.5

27. The potassium channel of claim 1 or 7, wherein the voltage-gated potassium channel is hERG.

28. A yeast cell comprising a deficiency in potassium uptake and a constitutively open voltage-gated potassium channel which allows said yeast cell to grow in the presence of media with very low potassium.

29. The yeast cell of claim 28, wherein the deficiency is due to a lack of TRK1 or TRK1 and TRK 2 potassium transporter activity.

30. The yeast cell of claim 28, wherein the deficiency is due to a lack of TRK1 and TRK 2 potassium transporter activity.

31. The yeast cell of claim 28 or 29, wherein the very low potassium concentration is about 2 mM or less.

32. The yeast cell of claim 28 or 29, wherein the constitutively open voltage-gated potassium channel comprises one or more mutations that are homologous to R400Q, R403Q, R409Q, or P513D in Kv1.5.

33. The yeast cell of claim 28 or 29, wherein the constitutively open voltage-gated potassium channel comprises two or more mutations that are homologous to R400Q, R403Q, R409Q, or P513D in Kv1.5.

34. The yeast cell of claim 28 or 29, wherein the constitutively open voltage-gated potassium channel comprises mutations homologous to R400Q, and P513D in Kv1.5.

35. The yeast cell of claim 28 or 29, wherein the constitutively open voltage-gated potassium channel comprises mutations homologous to R403Q, and P513D in Kv1.5.

36. The yeast cell of claim 28 or 29, wherein the constitutively open voltage-gated potassium channel comprises mutations homologous to R409Q, and P513D in Kv1.5.

37. The yeast cell of claim 28 or 29, wherein the constitutively open voltage-gated potassium channel comprises mutations homologous to R400Q, R403Q, and P513D in Kv1.5.

38. The yeast cell of claim 28 or 29, wherein the constitutively open voltage-gated potassium channel comprises mutations homologous to R400Q, R409Q, and P513D in Kv1.5.

39. The yeast cell of claim 28 or 29, wherein the constitutively open voltage-gated potassium channel comprises mutations homologous to R403Q, R409Q, and P513D in Kv1.5.

40. The yeast cell of claim 28 or 29, wherein the constitutively open voltage-gated potassium channel comprises mutations homologous to R400Q, R403Q, R409Q, and P513D In Kv1.5.

41. The yeast cell of claim 28 or 29, wherein the constitutively open voltage-gated potassium channel is a member of an ion channel family comprising Kv10, Kv11, and Kv12.

42. The yeast cell of claim 28 or 29, wherein the constitutively open voltage-gated potassium channel is a member of the Kv10 ion channel family.

43. The yeast cell of claim 28 or 29, wherein the constitutively open voltage-gated potassium channel is a member of the Kv11 ion channel family.

44. The yeast cell of claim 28 or 29, wherein the constitutively open voltage-gated potassium channel is a member of the Kv12 ion channel family.

45. The yeast cell of claim 28 or 29, wherein the constitutively open voltage-gated potassium channel is a member of an ion channel family comprising Kv1, Kv2, Kv3, and Kv4.

46. The yeast cell of claim 28 or 29, wherein the constitutively open voltage-gated potassium channel is a member of the Kv1 ion channel family.

47. The yeast cell of claim 28 or 29, wherein the constitutively open voltage-gated potassium channel is a member of the Kv2 ion channel family.

48. The yeast cell of claim 28 or 29, wherein the constitutively open voltage-gated potassium channel is a member of the Kv3 ion channel family.

49. The yeast cell of claim 28 or 29, wherein the constitutively open voltage-gated potassium channel is a member of the Kv4 ion channel family.

50. The yeast cell of claim 28 or 29, wherein the constitutively open voltage-gated potassium channel is Kv1.5 or hERG.

51. The yeast cell of claim 28 or 29, wherein the constitutively open voltage-gated potassium channel is Kv1.5.

52. The yeast cell of claim 28 or 29, wherein the constitutively open voltage-gated potassium channel is hERG.

53. A recombinant nucleic acid molecule comprising a nucleic add sequence encoding the potassium channel of claim 1.

54. A recombinant nucleic acid molecule comprising a promoter sequence operably linked to the nucleic acid molecule of claim 53.

55. A method comprising:
(a) providing a cell deficient in potassium uptake expressing the potassium channel of claim 1, wherein said cell has a high negative potential across the plasma membrane;
(b) growing the cell in very low potassium;
(c) adding a compound; and
(d) assaying the effect of the compound on the growth of the cell.

56. The method of claim 55, wherein the cell is a yeast cell.

57. The method of claim 56, wherein the yeast cell lacks TRK1 or TRK1 and TRK2 transporter activity.

58. The method of claim 56, wherein the yeast cell lacks TRK1 and TRK2 transporter activity.

59. The method of claim 57, wherein the yeast cell is S. cerevisiea.

60. The method of claim 55, wherein assaying the effect of the compound on the growth of the cell represents a procedure to determine the modulating activity of said compound on the potassium channel of claim 1.

61. The method of claim 60, wherein the modulating activity refers to the inhibition activity of said compound on the potassium channel of claim 1.
